(19) United States
(12) Patent Application Publication
(10) Pub. No.: US 2023/0355956 A1
(43) Pub. Date: Nov. 9, 2023

(54) Direct Cardiac Compression Device with Improved Durability

(71) Applicants: The Texas A&M University System, College Station, TX (US); CORINNOVA INCORPORATED, Houston, TX (US)

(72) Inventors: John C. Criscione, College Station, TX (US); Erica Christine Hord, Bryan, TX (US); Michael McDowall, Houston, TX (US)

(73) Assignees: The Texas A&M University System, College Station, TX (US); CorInnova Inc., Houston, TX (US)

(21) Appl. No.: 17/765,661

(22) PCT Filed: Dec. 14, 2020
(86) PCT No.: PCT/US2020/053920

§ 371 Date: Jun. 23, 2022

(51) Int. Cl. A61M 60/289; A61M 60/839; A61M 60/863

(52) U.S. Cl. CPC A61M 60/289; A61M 60/839; A61M 60/863; A61M 60/191

(57) ABSTRACT

The present invention provides a direct cardiac compression device comprising one or more passive chambers that taper from an aperture to an apex; one or more inflatable active pockets individually independently inflatable, wherein each of the one or more inflatable active pockets is connected to the one or more passive chambers at least partially from the aperture to the apex and wherein the each of the one or more inflatable active pockets does not tension the adjacent one or more inflatable active pockets upon inflation; and a frame in contact with the one or more active chambers to at least partially surround the one or more active pockets.

### CROSS-REFERENCE TO RELATED APPLICATIONS

This application is a National Stage of International Application No. PCT/US2020/053920, filed Oct. 2, 2020 which claims priority to and the benefit of U.S. Provisional Application No. 62/910,037, filed Oct. 3, 2019. The contents of each of which is incorporated by reference in their entirety.

### TECHNICAL FIELD OF THE INVENTION

The present invention relates in general to the field of heart assist devices, and more particularly, to methods and devices for assisting a heart with both short and an extended period of use of a direct cardiac compression device.

### STATEMENT OF FEDERALLY FUNDED RESEARCH

None.

INCORPORATION-BY-REFERENCE OF MATERIALS FILED ON COMPACT DISC

None.

### BACKGROUND OF THE INVENTION

Without limiting the scope of the invention, its background is described in connection with direct cardiac compression devices (DCCDs). The DCCD may be used for any length of time necessary—from short duration to longer extended periods of time ranging from hours to days and extend to weeks or even months. Previous DCCDs featured a circular single active chamber positioned to surround the heart and, in some instances, the DCCD may be partially divided into individual segments around the periphery thereof.

FIG. 1 is a cross-section of the direct cardiac compression device of the prior art. Shown in the drawing is only an active chamber 10. The active chamber 10 is divided into a plurality of individual inflatable active pockets 12. Each pocket 12 is in contact with adjacent pockets 12 at the connection points 18. Inflation of all pockets 12 at the same time from the same source of compressed air causes each pocket to expand in the mid-section and at the same time pull on the connection points 18 to bring them closer together. FIG. 2 is a side view of the direct cardiac compression device known in the prior art showing the connection points 18.

Since adjacent pockets 12 apply the same force on each connection point 18 but aimed at the opposite directions and tangential to the circular outline of the active chamber 12, the pull stress on each connection point 18 resulting from such arrangement is very high. Repeated application of such pull stress would cause premature failure of the device.

A second cause of premature failure is the excessive stress on the outer wall of the active chamber 10. Initial inflation with air causes the outer wall segments 14 to tense while the inner wall segments 16 are forced to move inwards and compress the heart located inside the active chamber 10. Tension stress on the outer segments 14 is high as the radius of curvature of the device is close to that of the cross-sectional shape of the heart. As a result, the flexible thin polymer material of the active chamber 10 has to withstand two mechanical loads at the same time, e.g., repeated flexing and tension stress. A combination of these two stresses leads to early failures and breaks in the continuity of the active chamber.

Another problem with DCCD designs in the prior art is the inability to follow a natural twisting motion of the heart. As seen in FIG. 1, the inflation of the prior art device causes a radial inward motion of the inner wall segments 16, while a natural motion of the heart muscle during heart contraction proceeds with a certain degree of twist, especially at the apex portion of the heart muscle. In a healthy heart, the apex may turn as much as 15 degrees while proceeding from end-diastole shape to end-systole shape during heart contraction. A twisting motion of the heart may cause tangential rubbing and slippage of the heart muscle against the inner wall of the device. Later in use, when the epicardial surface of the heart is presumably attached to the inner surface of the device, the twisting motion of the heart may cause excessive wrinkling of the device as the heart may drag the inner surface of the device along with it during every heart contraction, which may further increase the level of flexing stress on the device and cause its premature failure.

Furthermore, some prior art DCCD may feature an internal passive chamber located concentrically with and inside the active chamber 10 (not shown). Such passive chamber is used to fill the voids between the uneven and not exactly circular shape of the heart in cross-section and a perfectly round shape of the active chamber. In this case, the inflation of the pockets 12 of the active chamber 10 would cause compression of the heart indirectly, but rather through the passive chamber located in between the heart and the active chamber 10. The need exists for a more direct compression of the heart by the active chamber 10 to avoid additional resistance of compressing first the passive chamber inside thereof.

### DETAILED DESCRIPTION OF THE INVENTION

The object of the invention is to extend the period of use of a direct cardiac compression device beyond a few days, preferably up to 1 month or more. The DCCD may be used for any length of time necessary from short duration to longer extended periods of time ranging from hours to days and extend to weeks or even months. This allows to apply the device for additional groups of patients that require cardiac support for more than a day or two.

The present invention provides a direct cardiac compression device comprising one or more passive chambers that taper from an aperture to an apex; one or more inflatable active pockets individually independently inflatable, wherein each of the one or more inflatable active pockets is in contact with the one or more passive chambers at least partially from the aperture to the apex and wherein the each of the one or more inflatable active pockets does not tension the adjacent one or more inflatable active pockets upon inflation; and a frame in contact with the one or more active chambers to at least partially surround the one or more active chambers. In some embodiments the one or more inflatable active pockets are in contact with the one or more passive chambers through a direct connection. In some embodiments the one or more inflatable active pockets are in contact with the one or more passive chambers through an anchoring tab connected to the one or more inflatable active pockets and the one or more passive chambers.

The present invention provides a direct cardiac compression device comprising one or more passive chambers that taper from an aperture to an apex; one or more inflatable active pockets individually independently inflatable, wherein each of the one or more inflatable active pockets is connected to the one or more passive chambers at least partially from the aperture to the apex and wherein the each of the one or more inflatable active pockets does not tension the adjacent one or more inflatable active pockets upon inflation; and a frame in contact with the one or more active chambers to at least partially surround the one or more active chambers. The device further comprising a containment layer at least partially disposed around the direct cardiac compression device. The containment layer can extend partially around the device extending from the hub to one or more passive chambers, the one or more anchoring tabs, the one or more inflatable active pockets or even extend over the entire device covering the one or more passive chambers and extending back to the hub. The containment layer also can be formed from different materials over different areas, e.g., antiadhesion in one area and antimicrobial/antibacterial in another. In some embodiments each of the plurality of inflatable active pockets are connected to the support cone at the aperture. In some embodiments each of the plurality of inflatable active pockets are connected to the support cone at the aperture and the apex. In some embodiments each of the inflatable active pockets of the plurality of inflatable active pockets at least partially overlap. In some embodiments each of the plurality of inflatable active pockets are connected to the support cone by a spot weld, a seam weld, a weld line or a combination thereof. In some embodiments each of the plurality of inflatable active pockets are connected to the one or more anchoring tabs by a spot weld, a seam weld, a weld line or a combination thereof. In some embodiments each of the plurality of inflatable active pockets are connected to the one or more anchoring tabs by a spot weld, a seam weld, a weld line or a combination thereof. In some embodiments the plurality of inflatable active pockets includes 3-15 individual inflatable active pockets. In some embodiments the plurality of inflatable active pockets includes 5-10 individual inflatable active pockets. In some embodiments the plurality of inflatable active pockets has a heart-shaped contour. In some embodiments the device further comprises one or more fibers intercalated in the frame to provide support. In some embodiments the device further comprises a fiber reinforcement mesh in communication with the frame to provide support. In some embodiments the containment layer is connected to the one or more passive chambers. In some embodiments the containment layer is encloses the one or more passive chambers. In some embodiments the device further comprises a hub positioned at the apex and in operable communication with the one or more ports. In some embodiments the frame comprises a wire, a polymer, a shape memory material, a metal, an alloy, a composite or a combination thereof. In some embodiments the frame comprises an elastic energy storing element. In some embodiments the frame is embedded in the support cone, the plurality of inflatable active pockets or a combination thereof. In some embodiments the device further comprises one or more pharmacotherapies, stem cells, or other heart assist technologies to improve the function of a damaged or diseased heart. In some embodiments the device further comprises sensors embedded within the device capable of monitoring one or more of the following: temperature, pressure, EKG signal, conductivity. In some embodiments the device further comprises a containment layer positioned between the internal cone and a heart to aid in the removal of the direct cardiac compression device. In some embodiments the device further comprises a hub positioned at the apex in communication with the active pocket port, the passive pocket port or both. In some embodiments the containment layer is in contact with the hub and the plurality of passive pockets. In some embodiments the containment layer is not attached to the DCCD device making it removable without dislodging the containment layer. In some embodiments each of the plurality of inflatable active pockets are connected to the plurality of passive pockets by an anchoring tab. In some embodiments each of the one or more anchoring tabs extends at least partially from the aperture to the apex. In some embodiments device further comprises a second support structure positioned between the one or more passive chambers and the one or more inflatable active pockets.

The present invention provides a direct cardiac compression device adapted to be implanted in a patient suffering from heart failure and related cardiac pathologies, said direct cardiac compression device comprising one or more passive chambers that taper from an aperture to an apex; a plurality of inflatable active pockets connected to the one or more passive chambers, wherein the plurality of inflatable active pockets taper from the aperture to the apex and at least partially surround the one or more passive chambers and each of the plurality of inflatable active pockets is independently inflatable and wherein the each of the one or more inflatable active pockets does not tension the adjacent one or more inflatable active pockets when inflated; a support structure in contact with each of the plurality of inflatable active pockets, wherein the support structure extends at least partially from the aperture to the apex and each of the plurality of inflatable active pockets are connected to the support structure at one or more points; a frame in contact with the support structure to at least partially surround the support structure; and one or more ports in operable communication with each of the plurality of inflatable active pockets to independently inflate and deflate the plurality of inflatable active pockets and in operable communication with each of the one or more passive chambers to independently inflate and deflate the each of the one or more passive chambers. The device further comprising a containment layer at least partially disposed around the direct cardiac compression device. The containment layer can extend partially around the device extending from the hub to one or more passive chambers, the one or more anchoring tabs, the one or more inflatable active pockets or even extend over the entire device covering the one or more passive chambers and extending back to the hub. The containment layer also can be formed from different materials over different areas, e.g., antiadhesion in one area and antimicrobial/antibacterial in another. In some embodiments each of the plurality of inflatable active pockets are connected to the support cone at the aperture. In some embodiments each of the plurality of inflatable active pockets are connected to the support cone at the aperture and the apex. In some embodiments each of the inflatable active pockets of the plurality of inflatable active pockets at least partially overlap. In some embodiments each of the plurality of inflatable active pockets are connected to the support cone by a spot weld, a seam weld, a weld line or a combination thereof. In some embodiments each of the plurality of inflatable active pockets are connected to the one or more anchoring tabs by a spot weld, a seam weld, a weld line or a combination thereof. In some embodiments each of the plurality of inflatable active pockets are connected to the one or more anchoring tabs by a spot weld, a seam weld, a weld line or a combination thereof. In some embodiments the plurality of inflatable active pockets includes 5-15 individual inflatable active pockets. In some embodiments the plurality of inflatable active pockets includes 6-10 individual inflatable active pockets. In some embodiments the plurality of inflatable active pockets has a heart shaped contour. In some embodiments the device further comprises one or more fibers intercalated in the frame to provide support. In some embodiments the device further comprises a fiber reinforcement mesh in communication with the frame to provide support. In some embodiments the containment layer is connected to the one or more passive chambers. In some embodiments the containment layer is encloses the one or more passive chambers. In some embodiments the device further comprises a hub positioned at the apex and in operable communication with the one or more ports. In some embodiments the frame comprises a wire, a polymer, a shape memory material, a metal, an alloy, a composite or a combination thereof. In some embodiments the frame comprises an elastic energy storing element. In some embodiments the frame is embedded in the support cone, the plurality of inflatable active pockets or a combination thereof. In some embodiments the device further comprises one or more pharmacotherapies, stem cells, or other heart assist technologies to improve the function of a damaged or diseased heart. In some embodiments the device further comprises sensors embedded within the device capable of monitoring one or more of the following: temperature, pressure, EKG signal, conductivity. In some embodiments the device further comprises a containment layer positioned between the internal cone and a heart to aid in the removal of the direct cardiac compression device. In some embodiments the device further comprises a hub positioned at the apex in communication with the active pocket port, the passive pocket port or both. In some embodiments the containment layer is in contact with the hub and the plurality of passive pockets. In some embodiments the containment layer is removable. In some embodiments each of the plurality of inflatable active pockets are connected to the plurality of passive pockets by an anchoring tab. In some embodiments each of the one or more anchoring tabs extends at least partially from the aperture to the apex. In some embodiments device further comprises a second support structure positioned between the one or more passive chambers and the one or more inflatable active pockets.

The present invention provides a direct cardiac compression device that provides twist, said direct cardiac compression device comprising one or more passive pockets that taper from the aperture to the apex; a passive pocket port in operable communication with the one or more passive pockets to inflate and deflate the one or more passive pockets; a plurality of inflatable active pockets connected to the one or more passive pockets at a first attachment point, wherein each of the plurality of inflatable active pockets at least partially overlap the adjacent one or more inflatable active pockets without causing tension; an active pocket port in operable communication with the plurality of inflatable active pockets to individually inflate and deflate each of the one or more passive pockets to provide cardiac compression; a support structure positioned at least surrounding the plurality of inflatable active pockets and connected to the each of the plurality of inflatable active pockets at a second attachment point, wherein the first attachment point and the second attachment point result in a twisting motion of the plurality of inflatable active pockets during inflation and deflation; and a containment layer covering at least a portion of the direct cardiac compression device. The device further comprising a containment layer at least partially disposed around the direct cardiac compression device. The containment layer can extend partially around the device extending from the hub to one or more passive chambers, the one or more anchoring tabs, the one or more inflatable active pockets or even extend over the entire device covering the one or more passive chambers and extending back to the hub. The containment layer also can be formed from different materials over different areas, e.g., antiadhesion in one area and antimicrobial/antibacterial in another. In some embodiments each of the plurality of inflatable active pockets are connected to the support cone at the aperture. In some embodiments each of the plurality of inflatable active pockets are connected to the support cone at the aperture and the apex. In some embodiments each of the inflatable active pockets of the plurality of inflatable active pockets at least partially overlap. In some embodiments each of the plurality of inflatable active pockets are connected to the support cone by a spot weld, a seam weld, a weld line or a combination thereof. In some embodiments each of the plurality of inflatable active pockets are connected to the one or more anchoring tabs by a spot weld, a seam weld, a weld line or a combination thereof. In some embodiments each of the plurality of inflatable active pockets are connected to the one or more anchoring tabs by a spot weld, a seam weld, a weld line or a combination thereof. In some embodiments the plurality of inflatable active pockets includes 3-15 individual inflatable active pockets. In some embodiments the plurality of inflatable active pockets includes 5-10 individual inflatable active pockets. In some embodiments the plurality of inflatable active pockets has a heart shaped contour. In some embodiments the device further comprises one or more fibers intercalated in the frame to provide support. In some embodiments the device further comprises a fiber reinforcement mesh in communication with the frame to provide support. In some embodiments the containment layer is connected to the one or more passive chambers. In some embodiments the containment layer is encloses the one or more passive chambers. In some embodiments the device further comprises a hub positioned at the apex and in operable communication with the one or more ports. In some embodiments the frame comprises a wire, a polymer, a shape memory material, a metal, an alloy, a composite or a combination thereof. In some embodiments the frame comprises an elastic energy storing element. In some embodiments the frame is embedded in the support cone, the plurality of inflatable active pockets or a combination thereof. In some embodiments the device further comprises one or more pharmacotherapies, stem cells, or other heart assist technologies to improve the function of a damaged or diseased heart. In some embodiments the device further comprises sensors embedded within the device capable of monitoring one or more of the following: temperature, pressure, EKG signal, conductivity. In some embodiments the device further comprises a containment layer positioned between the internal cone and a heart to aid in the removal of the direct cardiac compression device. In some embodiments the device further comprises a hub positioned at the apex in communication with the active pocket port, the passive pocket port or both. In some embodiments the containment layer is in contact with the hub and the plurality of passive pockets. In some embodiments the containment layer is removable. In some embodiments each of the plurality of inflatable active pockets are connected to the plurality of passive pockets by an anchoring tab. In some embodiments each of the one or more anchoring tabs extends at least partially from the aperture to the apex. In some embodiments device further comprises a second support structure positioned between the one or more passive chambers and the one or more inflatable active pockets.

The present invention provides a method of treating a suffering one or more symptoms of heart failure comprising the steps of providing a direct cardiac compression device comprising one or more passive chambers that taper from an aperture to an apex; a plurality of inflatable active pockets connected to the one or more passive chambers, wherein the plurality of inflatable active pockets taper from the aperture to the apex and at least partially surround the one or more passive chambers and each of the plurality of inflatable active pockets is independently inflatable and wherein the each of the one or more inflatable active pockets does not tension the adjacent one or more inflatable active pockets when inflated; a support structure in contact with each of the plurality of inflatable active pockets, wherein the support structure extends at least partially from the aperture to the apex and each of the plurality of inflatable active pockets are connected to the support structure at one or more points; a frame in contact with the support structure to at least partially surround the support structure; and one or more ports in operable communication with each of the plurality of inflatable active pockets to independently inflate and deflate the plurality of inflatable active pockets and in operable communication with each of the one or more passive chambers to independently inflate and deflate the each of the one or more passive chambers; and implanting the direct cardiac compression device in a patient suffering one or more symptom of heart failure or related cardiac pathologies. The device further comprising a containment layer at least partially disposed around the direct cardiac compression device. The containment layer can extend partially around the device extending from the hub to one or more passive chambers, the one or more anchoring tabs, the one or more inflatable active pockets or even extend over the entire device covering the one or more passive chambers and extending back to the hub. The containment layer also can be formed from different materials over different areas, e.g., antiadhesion in one area and antimicrobial/antibacterial in another. In some embodiments each of the plurality of inflatable active pockets are connected to the support cone at the aperture. In some embodiments each of the plurality of inflatable active pockets are connected to the support cone at the aperture and the apex. In some embodiments each of the inflatable active pockets of the plurality of inflatable active pockets at least partially overlap. In some embodiments each of the plurality of inflatable active pockets are connected to the support cone by a spot weld, a seam weld, a weld line or a combination thereof. In some embodiments each of the plurality of inflatable active pockets are connected to the one or more anchoring tabs by a spot weld, a seam weld, a weld line or a combination thereof. In some embodiments each of the plurality of inflatable active pockets are connected to the one or more anchoring tabs by a spot weld, a seam weld, a weld line or a combination thereof. In some embodiments the plurality of inflatable active pockets includes 5-15 individual inflatable active pockets. In some embodiments the plurality of inflatable active pockets includes 6-10 individual inflatable active pockets. In some embodiments the plurality of inflatable active pockets has a heart shaped contour. In some embodiments the device further comprises one or more fibers intercalated in the frame to provide support. In some embodiments the device further comprises a fiber reinforcement mesh in communication with the frame to provide support. In some embodiments the containment layer is connected to the one or more passive chambers. In some embodiments the containment layer is encloses the one or more passive chambers. In some embodiments the device further comprises a hub positioned at the apex and in operable communication with the one or more ports. In some embodiments the frame comprises a wire, a polymer, a shape memory material, a metal, an alloy, a composite or a combination thereof. In some embodiments the frame comprises an elastic energy storing element. In some embodiments the frame is embedded in the support cone, the plurality of inflatable active pockets or a combination thereof. In some embodiments the device further comprises one or more pharmacotherapies, stem cells, or other heart assist technologies to improve the function of a damaged or diseased heart. In some embodiments the device further comprises sensors embedded within the device capable of monitoring one or more of the following: temperature, pressure, EKG signal, conductivity. In some embodiments the device further comprises a containment layer positioned between the internal cone and a heart to aid in the removal of the direct cardiac compression device. In some embodiments the device further comprises a hub positioned at the apex in communication with the active pocket port, the passive pocket port or both. In some embodiments the containment layer is in contact with the hub and the plurality of passive pockets. In some embodiments the containment layer is removable. In some embodiments each of the plurality of inflatable active pockets are connected to the plurality of passive pockets by an anchoring tab. In some embodiments each of the one or more anchoring tabs extends at least partially from the aperture to the apex.

The present invention provides a direct cardiac compression device comprising one or more passive chambers that taper from an aperture to an apex; a second support structure in contact with the one or more passive chambers; a plurality of inflatable active pockets in contact with the second support structure and optionally with the to the one or more passive chambers, wherein the plurality of inflatable active pockets taper from the aperture to the apex and at least partially surround the second support structure and each of the plurality of inflatable active pockets is independently inflatable and wherein the each of the one or more inflatable active pockets does not tension the adjacent one or more inflatable active pockets when inflated; a support structure in contact with each of the plurality of inflatable active pockets, wherein the support structure extends at least partially from the aperture to the apex and each of the plurality of inflatable active pockets are connected to the support structure at one or more points; a frame in contact with the support structure to at least partially surround the support structure; and one or more ports in operable communication with each of the plurality of inflatable active pockets to independently inflate and deflate the plurality of inflatable active pockets and in operable communication with each of the one or more passive chambers to independently inflate and deflate the each of the one or more passive chambers.

### DESCRIPTION OF THE DRAWINGS

For a more complete understanding of the features and advantages of the present invention, reference is now made to the detailed description of the invention along with the accompanying figures and in which:

FIG. 1 is a top cross-section view of a direct cardiac compression device known in the prior art;

FIG. 2 is a side view of a direct cardiac compression device known in the prior art;

FIG. 3 is a side view of the direct cardiac compression device of the present invention;

FIG. 4 is a top view of one embodiment of the direct cardiac compression device of the present invention;

FIG. 5 is a cross-sectional view of another embodiment of the direct cardiac compression device 10 of the present invention;

FIG. 6 is a cut through view of a direct cardiac compression device showing the attachment using a line weld; and

FIG. 7 shows an exemplary cross-sectional side view of a device assembly.

FIG. 8 is a cross-sectional view

FIG. 9 is a top view of another embodiment of the direct cardiac compression device 10 of the present invention.

FIG. 10 is a cross-sectional view of another embodiment of the direct cardiac compression device of the present invention.

FIG. 11 is a top view of another embodiment of the direct cardiac compression device 10 of the present invention.

FIG. 12 is a top view of a portion of another embodiment of the direct cardiac compression device of the present invention.

FIG. 13 is a top view of another embodiment of a portion of the direct cardiac compression device of the present invention.

FIG. 14 is a top view of another embodiment of a portion of the direct cardiac compression device of the present invention.

FIG. 15 illustrates a top view of another embodiment of a portion of the direct cardiac compression device showing individual active anchoring tabs connected to the adjacent active anchoring tab.

FIG. 16 illustrates a top view of another embodiment the direct cardiac compression device having the frame positioned over the active chambers.

FIG. 17 illustrates a top view of another embodiment the direct cardiac compression device having the containment layer positioned over the frame.

FIG. 18 is a top view of the frame.

FIG. 19 is a top view of the frame having the supports connecting the frame. FIG. 20 is a side view of the frame having the supports connecting the frame. FIG. 21 is a side view of the direct cardiac compression device.

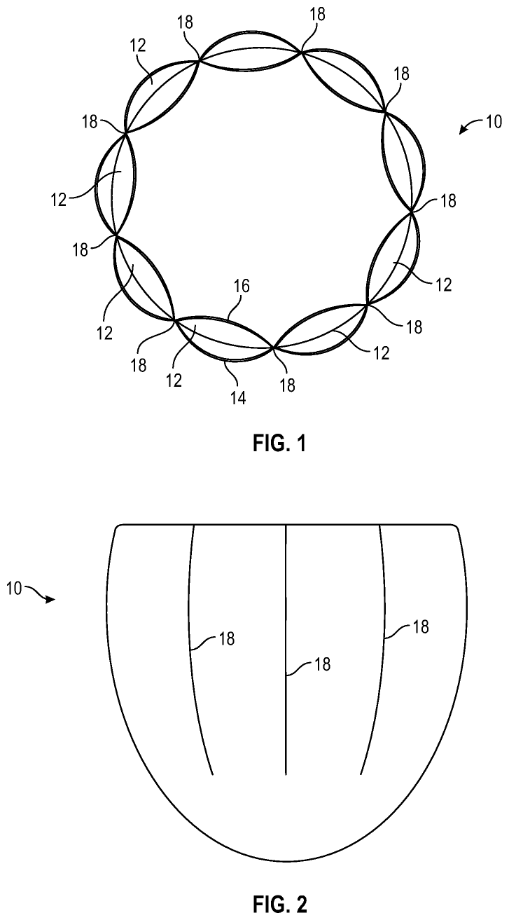

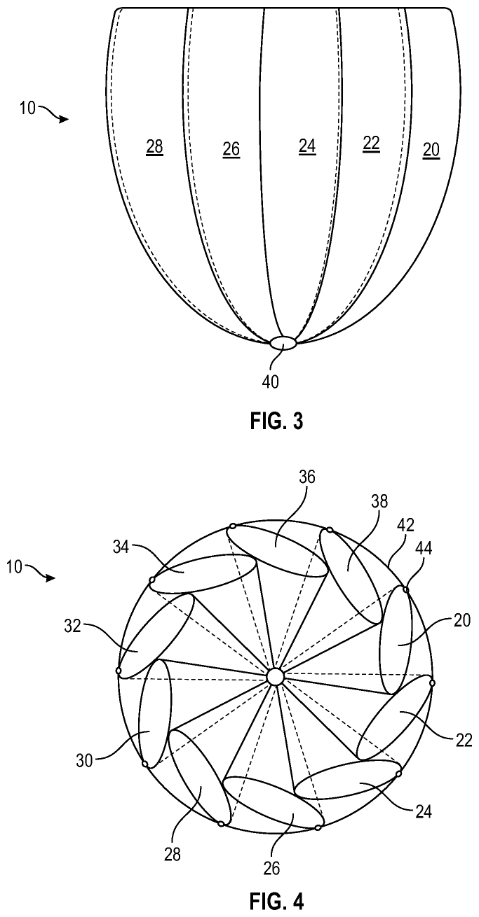

### DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention.

As used herein, a “biomedical material” denotes a material which is physiologically inert to avoid rejection or other negative inflammatory response.

As used herein, the term “joining line” denotes any manner of joining 2 materials, including but not limited to heating, welding, gluing, bonding, adhesives, melting, tacking, etc.

As used herein, “thin polymer film,” “polymer film,” polymer” and “film” denoted a material that is substantially biocompatible, fluid-impermeable and substantially inelastic. For example, at least a portion of the device may be made from elastomeric polyurethane, latex, polyetherurethane, polycarbonateurethane, silicone, polysiloxaneurethane, hydrogenated polystyrene-butadiene copolymer, ethylene-propylene and dicyclopentadiene terpolymer, hydrogenated poly(styrene-butadiene) copolymer, poly(tetramethylene-ether glycol) urethanes, poly(hexamethylenecarbonate-ethylenecarbonate glycol) urethanes and combinations thereof.

As used herein, “fiber reinforcement mesh” or “fiber reinforcement layer” denotes any fiber and any configuration. For example, the fiber configuration may be a mesh or a weave of fiber in any number of thickness or orientation. In addition, the fiber reinforcement layer includes individual fibers or bundles of fibers or a non-mesh thicker layer. The fiber reinforcement layer may be one or more layers and may include layers of similar and dissimilar design, e.g., a woven mesh layer with a layer of individual fibers oriented in a first direction with another layer of individual fiber orientated in a second direction.

As used herein, “cone,” “external cone,” “internal cone,” “support cone,” and “support structure” are used interchangeably to denote a support structure.

The present invention provides a direct cardiac compression device designed for use for both short and extended periods of time from a few days to one or more months. The present invention allows the DCCD to be used to treat additional groups of patients that require cardiac support for more than a day or two. The present invention allows the DCCD may be used for any length of time necessary from short duration to longer extended periods of time ranging from hours to days and extend to weeks or even months.

The present invention provides a direct cardiac compression device having an active chamber comprising a plurality of independently assembled inflatable active pockets which do not cause a tension stress on adjacent active pockets upon inflation.

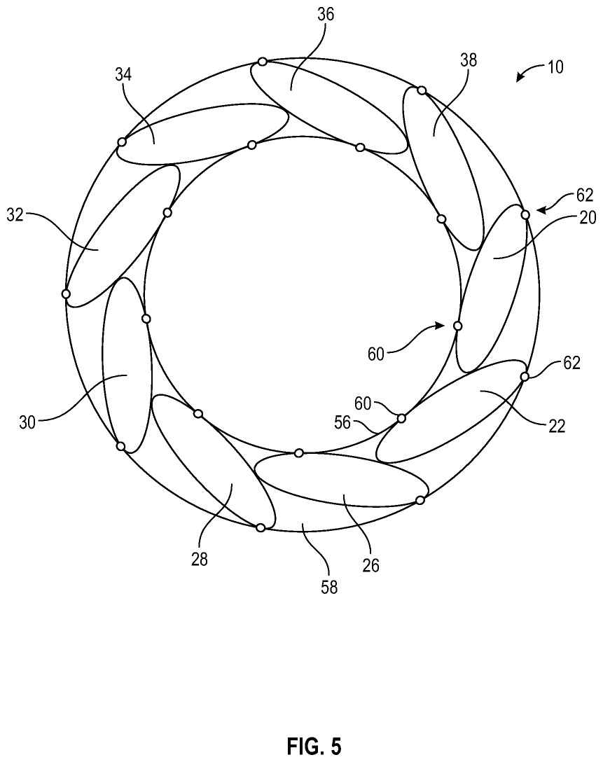

FIG. 3 is a side view of the direct cardiac compression device of the present invention. FIG. 4 is a top view of the direct cardiac compression device of the present invention. The direct cardiac compression device 10 includes the new active chamber, which in this case comprises a plurality of individual inflatable active pockets 20-38 (the number of pockets may vary from about 3 to about 15) extending from a common hub 40 and positioned in pneumatic communication with each other and with the source of compressed air and vacuum operatively attached to the hub 40 (not shown). In addition, the hub 40 may be a bundle of individual tubes. Each inflatable pocket 20-38 extends along the projected cone representing the shape of the heart towards the heart base at the top of FIG. 3. To assure proper arrangement of the inflatable active pockets 20-38 and prevent shifting around while in use, an external cone 42 is positioned around the exterior of the inflatable active pockets 20-38 and may be made from the same thin polymer film as the inflatable active pockets 20-38 or from another polymer material. Individually, each pocket may be attached to the external cone 42 at a single point 44 or along a portion or the entire length in the form of a joining line extending from the point 44 downwards and towards the hub 80. In FIG. 3, each inflatable active pocket 20-38 is only attached to the external cone 42 and not to each adjacent inflatable pocket 20-38. Individual inflatable active pockets 20-38 may be positioned to overlap each adjacent inflatable pocket 20-38 as shown in FIG. 4 but this is an optional feature of the design of the device. In use, inflation of each pocket 20-38 causes individual compression of the heart at the respective location of each pocket as the outer external cone 42 resists the tension load applied by all of the inflatable active pockets 20-38. Importantly, the tangential tension stress on each pocket is removed which promotes a longer life of the polymer film.

The present invention provides a direct cardiac compression device 10 having new active chambers and an external cone 42 that provides a uniform distribution of tensile stresses and avoids creating one or more stress concentration points. The present invention also provides a fiber reinforcement layer outside the inflatable active pockets which is configured to absorb and resist the outwards expansion of the device as the active chamber is inflated. In addition, the fiber reinforcement layer may be incorporated outside or incorporated into the outer wall of the inflatable active pockets. The external cone 42 is designed and configured to absorb a plurality of external forces applied by the inflatable active pockets 20-38 without creating one or more stress concentration points. In contrast to the prior art devices, the present invention provides an external cone 42 that receives the applied forces and limits the forces seen by the inflatable active pockets themselves as was the case with the prior art devices.

The present invention provides a direct cardiac compression device 10 having the ability to separate the wire frame from the active chamber. At least in some devices of the prior art, the wire frame was built into the inflatable segments of the active chamber. In the direct cardiac compression device 10 of the present invention, the wire frame may be positioned outside the active chamber allowing for greater flexibility in the design of both the active chamber and the wire frame itself. A number of further useful improvements are contemplated to be a part of the direct cardiac compression device 10 of the present invention.

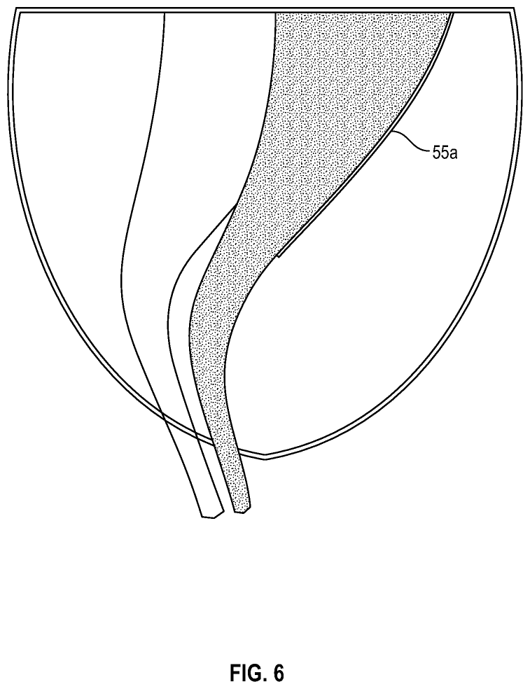

FIG. 5 is a cross-sectional view of another embodiment of the direct cardiac compression device 10 of the present invention. The direct cardiac compression device 10 includes a plurality of inflatable active pockets 20-38 (eight in this case) are positioned between an external cone 42 and an internal cone 56. Each inflatable pocket is attached on one side to the internal cone 56 at points 60 on this cross-sectional drawing. The other side of the inflatable pocket 20 is attached to the external cone 42 at the points 62. FIG. 6 is a cut through view of a direct cardiac compression device showing the attachment using a line weld. Points 62 represent a chamber weld line as seen in FIG. 6. Similarly, points 60 represent a set of corresponding chamber weld lines on the internal cone 56 (not shown). Using this arrangement allows the direct cardiac compression device to retain the plurality of inflatable active pockets 20-38 between two continuous layers of a thin polymer film forming the internal cone 56 and the external cone 52. Filling the space 58 with saline allows formation of a passive fluid-filled space in between the internal cone 56 and the external cone 52 to provide both passive and active chambers. In this case, however, the passive chambers are not located solely inside the active chambers. Rather, the fluid is free to fill the space between the inflatable active pockets 20-38 so as to make a tight fit between the heart and the external cone 42. The space 58 between the internal cone 56 and the external cone 52 form a passive chamber that may be configured as needed by adjusting the pressure within the space 58. As an alternative, one or more passive chambers may be positioned inside the inflatable active pockets or inside the internal cone. A fluid may be added to the space 58 to adjust the fitment around the heart. The fluid may be a gas, a liquid or a combination thereof.

Another embodiment of the direct cardiac compression device provides a twist during inflation. As the inflatable pocket expands during inflation, a pair of attachment points 62 and 60 are moving closer to each other. For a vertically oriented direction of the inflatable pocket 20 as seen in FIG. 3 for example, this movement of the attachment point 62 towards the attachment point 60 causes a relative twist of the internal cone 56 inside the external cone 42. Orienting at least a portion of the inflatable pocket 20 diagonally, as seen in FIG. 6 for example, leads to a relative twisting motion of the internal cone 56 in a manner similar to the motion of the heart. Adjusting the extend of angular orientation of the inflatable pocket welding lines for both sides of each inflatable pocket 20-38 may be used to achieve a more natural twisting motion of the internal cone 56 matching that of the native heart epicardial surface. In addition, at least a portion of the inflatable pocket 20 may be oriented diagonally, horizontally, vertically or a combination thereof.

A further advantage of angular direction of the inflatable active pockets is that it may facilitate an easier compression of the device as it is being drawn into a delivery tube. In addition, a lack of attachment of the inflatable active pockets at the location adjacent the hub may also promote greater flexibility and the ability to compress the device to a smaller size prior to deployment.

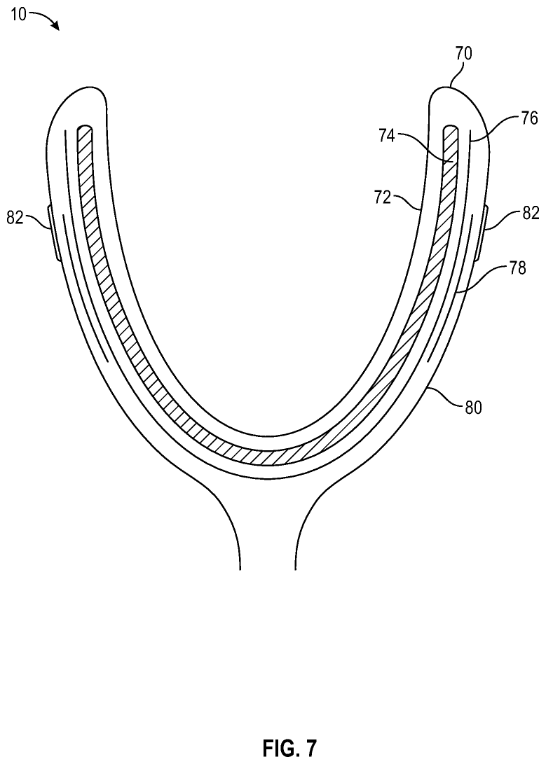

FIG. 7 shows an exemplary cross-sectional side view of a device assembly. Shown as position 74 is a subassembly of the active and passive chambers as described above, featuring a plurality of inflatable active pockets operably connected to a source of compressed air (not shown). A wireframe 76 may be positioned outside the active chamber 74 and can be made using conventional NiTi wire. The skilled artisan will recognize that other metals, alloys, polymers and combinations thereof can be used. The wire frame 76 may be positioned inside the internal cone, between the internal cone and the external cone, and outside the external cone as the invention is not limited in this regard.

A fiber reinforcement layer 78 may be positioned further outside the wireframe 76 or inside the wireframe 76 next to the active chamber 74. This fiber reinforcement layer 78 may be made using individual strands of fiber such as polyester or nylon ribbons, or a polymer mesh configured to contain outward expansion of the active chamber during inflation of the pockets 20-38. In embodiments, the mesh 78 may expand fully or partially from the hub 40 to the top of the device. FIG. 7 shows an example of a partial coverage of the device with the mesh layer 78 at the middle section of the device.

The fiber reinforcement layer 78 may be incorporated in the external cone 42 such as embedded in the polymer film of the external cone 42, or by other attachment means secured to the external surface thereof. In further embodiments, the fiber reinforcement layer 78 may be attached to the inside surface or the outside surface, or attached to both or in further embodiments can be weaved therein. In still other embodiments, the fiber reinforcement layer 78 may be integrated into the inside surface or the outside surface, or both. Still other embodiments may be a combination of attachment to the inside surface or the outside surface and the integration into the inside surface or the outside surface, allowing numerous combinations.

One advantage of positioning the fiber reinforcement layer 78 outside or integral with the wireframe 76 is to limit the outward motion of the wireframe upon expansion of the inflatable active pockets, whereby reducing the flexing load on the NiTi wire and improving longevity thereof. In other embodiments, the fiber reinforcement layer 78 may be formed integrally with the external portion 80 of the containment layer 70.

Individual fibers or strands forming together a mesh 108 need to be strong enough to withstand together the tensile stress of expanding inflatable active pockets, whereby directing their expansion inwards and towards the heart. On the other hand, the fibers need to be made as thin and flexible as possible so as not to increase the size of the delivery sheath and maximize device flexibility during insertion and removal procedures.

Containment layer 70 may include an internal portion 72 designed to separate the device from the heart and the external portion 80 located outside the device and separating it from the pericardium. A source of continuous low-level vacuum aspiration may be operatively connected to the inside space of the containment layer 70 so that any air leak can be quickly aspirated so as to reduce the risk of a heart tamponade.

A further aim of the containment layer 70 is to allow device replacement in case of a leak. If the original device is retained inside the patient for a few days, the surface of the containment layer may adhere to the tissues of the patient—either the heart inside thereof or the pericardium outside thereof—or both. In this case, as the device is not attached to the containment layer 70, it can still be removed from within the internal space inside the containment layer 70—and replaced with another device, which in this case may be directly deployed inside the containment layer 70. In addition, a suitable lubricant or other anti-adhesion substance may be used to separate the active chamber from the containment layer to minimize tissue adhesion and allow for a free movement of the active chamber film against the thin film of the containment layer. Specific suitable lubricant may be liquid, fluid, powder, etc. and specific examples include silicon and Teflon powders. In addition, certain embodiments of the present invention may have leads, electrodes or electrical connections incorporated into the device. When present, they may be made from noble metals (e.g., gold, platinum, rhodium and their alloys) or stainless steel. In addition, ordinary pacemaker leads and defibrillation leads can be incorporated into the present invention to provide cardiac pacing or defibrillation. The containment layer may remain in place in the body after removal of active chamber and wire frame to maintain access to external surface of heart during tissue scaring and healing for potential future device implantation

One, two or more electrodes 82 may be positioned inside or outside the device and may be located on a tissue-contacting surface of the containment layer 70—to be configured to sense ECG signal either directly from the external surface of the heart or from the internal surface of the pericardium (as seen in FIG. 7). The ECG signals may be recorded, stored or transmitted to aid in timing of the device inflation, diagnostics, treatments or to be used as inputs for other devices.

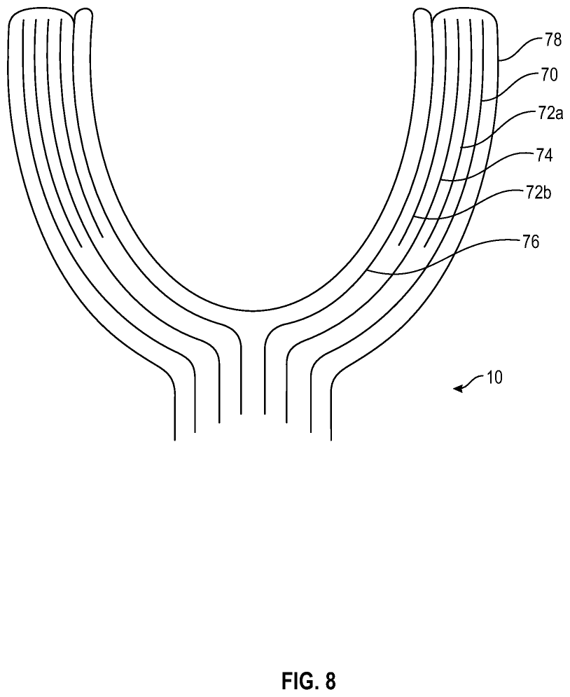

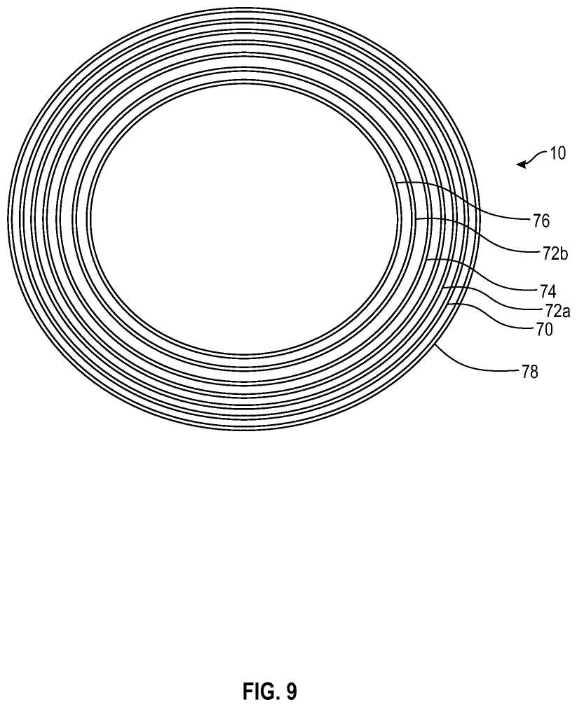

FIG. 8 is a cross-sectional view and FIG. 9 is a top view of another embodiment of the direct cardiac compression device 10 of the present invention. The direct cardiac compression device 10 includes a frame 70 in contact with a first securement cone 72a that extends at least partially down the frame 70. An active chamber layer 74 is in contact with a first securement cone 72a and a second securement cone 72b that extends at least partially down the active chamber layer 74. A passive layer 76 is in contact with the second securement cone 72b which that extends at least partially over the passive layer 76. The passive layer 76 and the active chamber 74 may be connected through a circumferential basal weld. A containment layer 78 houses the direct cardiac compression device 10 extending from the passive layer 76 to encapsulate the frame 70.

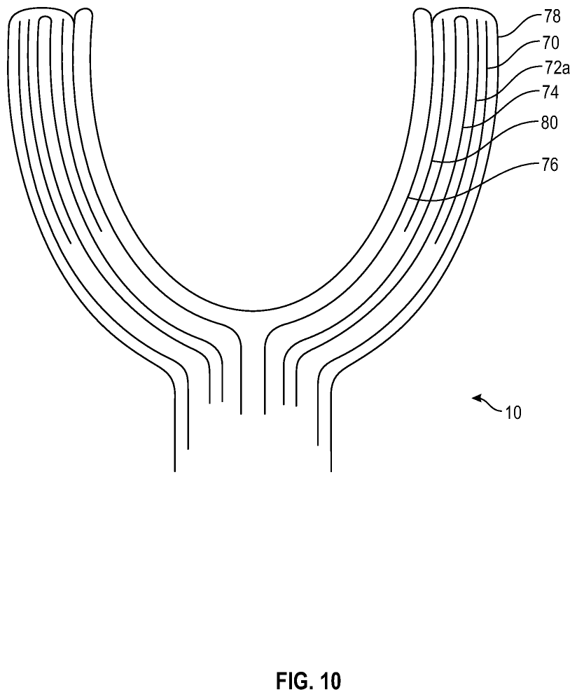

FIG. 10 is a cross-sectional view of another embodiment of the direct cardiac compression device 10 of the present invention. The direct cardiac compression device 10 includes a frame 70 in contact with a first securement cone 72a that extends at least partially down the frame 70. An active chamber layer 74 is in contact with an active anchoring tab 80 that extends and connects to a passive layer 70. The active chamber layer 74 may include numerous active chambers ranging from 2, 3, 4, 5, 6, 7, 8, 9, 10, 11, 12, 13, 14, 15, 16, 17, 18 or more. A containment layer 78 houses the direct cardiac compression device 10 extending from the passive layer 76 to encapsulate the frame 70.

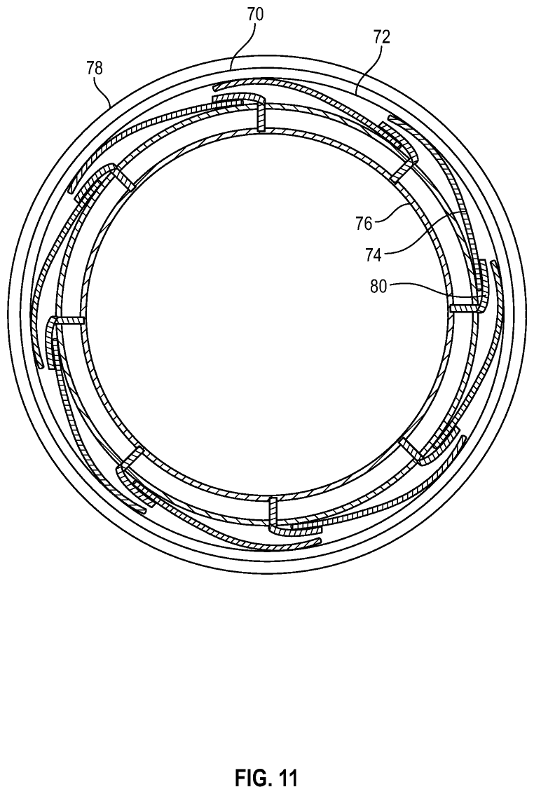

FIG. 11 is a top view of another embodiment of the direct cardiac compression device 10 of the present invention. The direct cardiac compression device 10 includes a frame 70 in contact with a first securement cone 72a that extends at least partially down the frame 70. An active chamber layer 74 is in contact with an active anchoring tab 80 that extends and connects to a passive layer 76. In this embodiment the active chamber layer 74 includes 8 active chambers. Each of the 8 active chambers is individually connected to the passive layer through an individual active anchoring tab 80. The active anchoring tab 80 may extend the entire length of the active chamber layer 74 and the passive layer 76 or may extend only partially the length. The passive layer 76 may include individual passive chambers. In some embodiments, the individual passive chambers are formed by connecting the sides of adjacent individual passive chambers. In some embodiments the active anchoring tab 80 is positioned between adjacent individual passive chambers and then connected together. A containment layer 78 houses the direct cardiac compression device 10 extending from the passive layer 70 to encapsulate the frame 70. In an alternate embodiment, the active chamber layer 74 is in contact directly with a passive layer 76. Each of the 8 active chambers is individually connected to the passive layer through and may extend the entire length of the active chamber layer 74 and the passive layer 76 or may extend only partially the length.

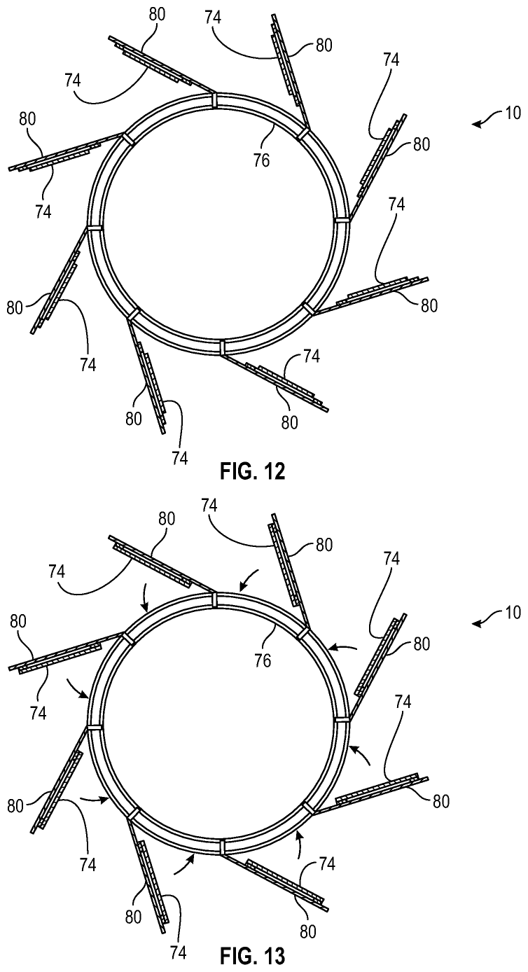

FIG. 12 is a top view of another embodiment of a portion of the direct cardiac compression device 10 of the present invention. The direct cardiac compression device 10 includes the active chamber layer 74 which has 8 active chambers 82 each of which is connected to an active anchoring tab 80 that extends and connects to a passive layer 76. In this embodiment the passive layer 76 is divided into 8 individual passive chambers.

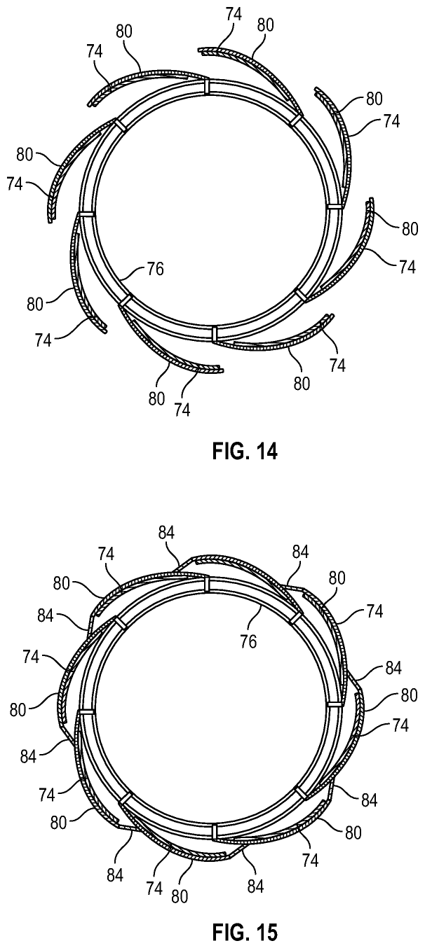

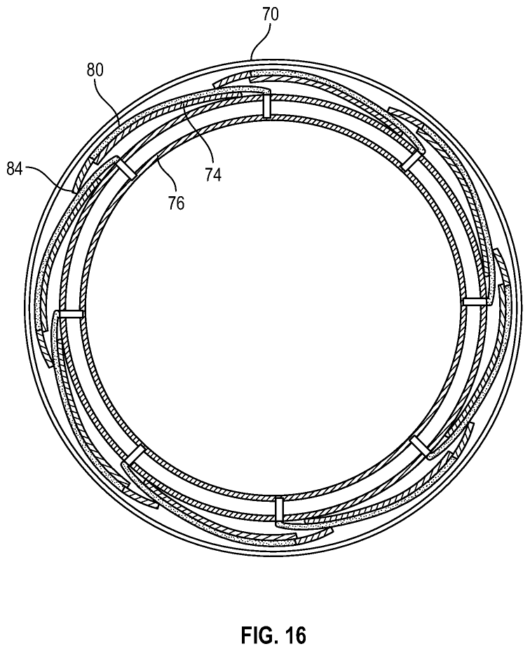

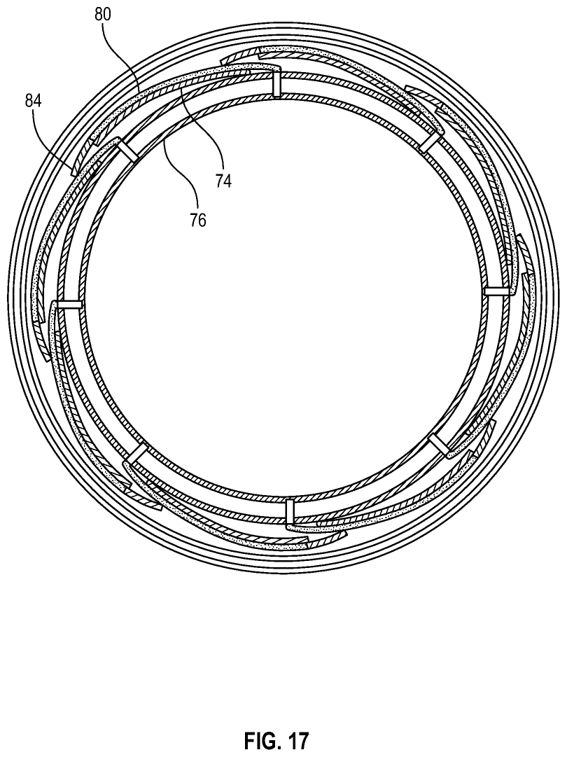

FIG. 13 is a top view of another embodiment of a portion of the direct cardiac compression device 10 of the present invention. The direct cardiac compression device 10 includes the active chamber layer 74 which has 8 active chambers 82 each of which is connected to an active anchoring tab 80 that extends and connects to a passive layer 76. In this embodiment the active anchoring tab 80 allows each of the active chambers 82 to fold and collapse as shown in FIG. 14. In this embodiment the passive layer 76 is divided into 8 individual passive chambers. FIG. 15 illustrates the individual active anchoring tab 80 can also be connected to the adjacent active anchoring tab 80 as illustrated. The connection is shown as 84. FIG. 16 illustrates a top view of another embodiment the direct cardiac compression device 10 having the frame positioned over the active chambers 82. FIG. 17 illustrates a top view of another embodiment the direct cardiac compression device 10 having the containment layer positioned over the frame 70.

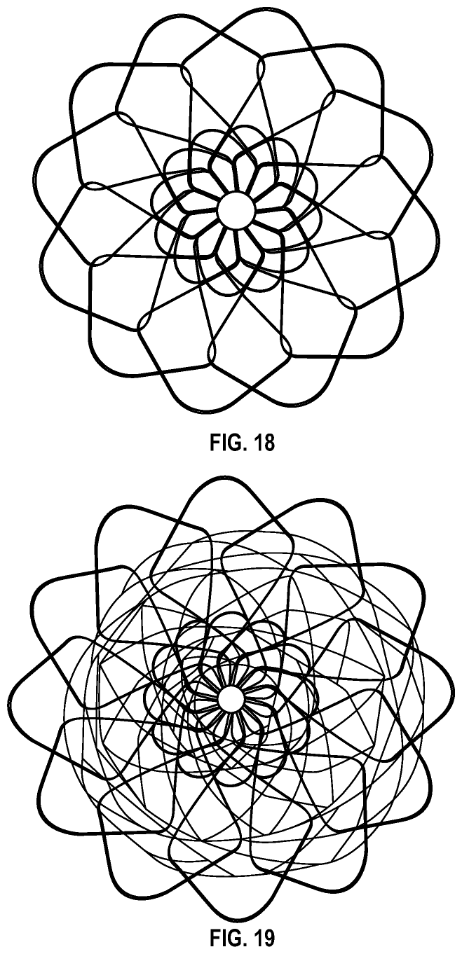

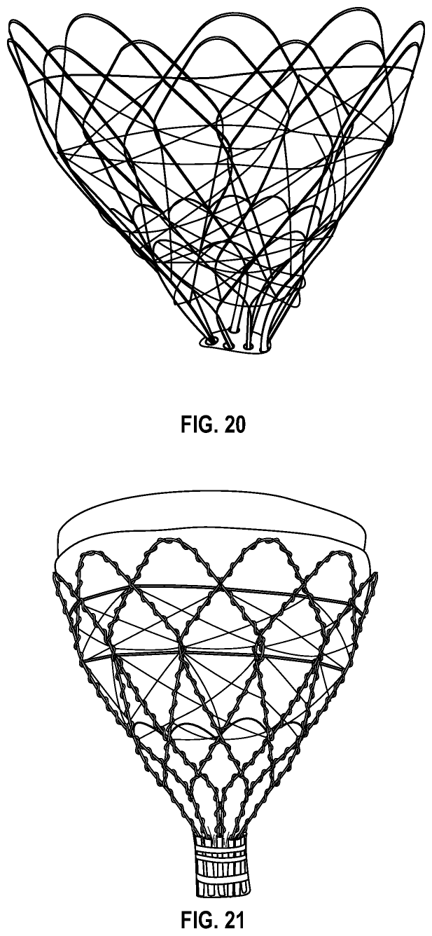

FIG. 18 is a top view of the frame 70. FIG. 19 is a top view of the frame 70 having the supports connecting the frame 70. FIG. 20 is a side view of the frame 70 having the supports connecting the frame 70. FIG. 21 is a side view of the direct cardiac compression device.

Generally, when a material is implanted in the body, the body recognizes the presence of the foreign material and triggers an immune defense system to eject and destroy the foreign material. This results in edema, inflammation of the surrounding tissue and biodegradation of the implanted material. As a result, the present invention is at least partially comprised of biomedical implantable material. Examples of suitable, biocompatible, biostable, implantable materials used to fabricate the present invention include, but are not limited to, polyetherurethane, polycarbonateurethane, silicone, polysiloxaneurethane, hydrogenated polystyrene-butadiene copolymer, ethylene-propylene and dicyclopentadiene terpolymer, and/or hydrogenated poly(styrene-butadiene) copolymer, poly(tetramethylene-ether glycol) urethanes, poly(hexamethylenecarbonate-ethylenecarbonate glycol) urethanes and combinations thereof. In addition, the present invention may be reinforced with filaments made of a biocompatible, biostable, implantable polyamide, polyimide, polyester, polypropylene, and/or polyurethane.

The material used in the construction of the present invention minimizes the incidence of infection associated with medical device implantation such as entercoccus, pseudomonas auerignosa, staphylococcus and staphylococcus epidermis infections. Embodiments of the present invention include bioactive layers or coatings to prevent or reduce infections. For example, bioactive agents may be implanted, coated or disseminated on the present invention and include antimicrobials, antibiotics, antimitotics, antiproliferatives, antisecretory agents, non-steroidal anti-inflammatory drugs, immunosuppressive agents, antipolymerases, antiviral agents, antibody targeted therapy agents, prodrugs, free radical scavengers, antioxidants, biologic agents or combinations thereof Antimicrobial agents include but are not limited to benzalkoniumchloride, chlorhexidine dihydrochloride, dodecarbonium chloride and silver sufadiazine. Generally, the amount of antimicrobial agent required depends upon the agent; however, concentrations range from 0.0001% to 5.0%.

Certain embodiments of the present invention can be used in conjunction with cardiac stem cell therapies. Stem cells used for cardiac regeneration therapy include but are not limited to stem cells derived from embryonic stem cells, somatic stem cells taken from bone marrow, progenitor cells from cardiac tissue, autologous skeletal myoblasts from muscle tissue, hematopoietic stem cells, mesenchymal stem cells, and endothelial precursor cells. The present invention can also be used in combination naturally occurring cardiac stem cells. Transplanted stem cells may be injected directly into cardiac tissue including, infarcted regions, cardiac scar tissue, borderzones, or healthy cardiac tissue. Transplanted stem cells may also be injected systemically feeding regions of cardiac tissue and may migrate to regions of the damaged or diseased heart and engraft to regions of the damaged or diseased heart. Transplanted stem cells may also provide diffusible products to regions of the damaged or diseased heart.

The direct cardiac compression device of the present invention includes inflatable compartments connected to a fluid pressure source through an inlet port and an outlet port. The device is inflated with a positive pressure during systole and deflated (via suction) during diastole. Other configurations and multiple connections are also possible depending on the particular application and configuration of the direct cardiac compression device.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

1. A direct cardiac compression device comprising
one or more passive chambers that taper from an aperture to an apex;
one or more inflatable active pockets individually independently inflatable, wherein each of the one or more inflatable active pockets is connected to the one or more passive chambers at least partially from the aperture to the apex and wherein the each of the one or more inflatable active pockets does not tension the adjacent one or more inflatable active pockets upon inflation; and
a frame in contact with the one or more active pockets to at least partially surround the one or more active pockets.

2. A direct cardiac compression device comprising
one or more passive chambers that taper from an aperture to an apex;
a plurality of inflatable active pockets connected to the one or more passive chambers, wherein the plurality of inflatable active pockets taper from the aperture to the apex and at least partially surround the one or more passive chambers and each of the plurality of inflatable active pockets is independently inflatable and wherein the each of the one or more inflatable active pockets does not tension the adjacent one or more inflatable active pockets when inflated;
a support structure in contact with each of the plurality of inflatable active pockets, wherein the support structure extends at least partially from the aperture to the apex and each of the plurality of inflatable active pockets are connected to the support structure at one or more points;
a frame in contact with the support structure to at least partially surround the support structure; and
one or more ports in operable communication with each of the plurality of inflatable active pockets to independently inflate and deflate the plurality of inflatable active pockets and in operable communication with each of the one or more passive chambers to independently inflate and deflate the each of the one or more passive chambers.

3. The device of claim 1, wherein each of the plurality of inflatable active pockets are connected to the plurality of passive pockets by an anchoring tab.

4. The device of claim 3, wherein the anchoring tab extends at least partially from the aperture to the apex.

5. The device of claim 1, further comprising a containment layer at least partially disposed around the direct cardiac compression device.

6. The device of claim 1, wherein each of the plurality of inflatable active pockets are connected to the support structure at the aperture or at the aperture and the apex.

7. The device of claim 1, wherein each of the inflatable active pockets of the plurality of inflatable active pockets at least partially overlap.

8. The device of claim 1, wherein each of the plurality of inflatable active pockets are connected by a spot weld, a seam weld, a weld line or a combination thereof.

9. The device of claim 1, wherein the plurality of inflatable active pockets includes 3-15 individual inflatable active pockets, preferably 5-10 individual inflatable active pockets and more preferably 7-9 individual inflatable active pockets.

10. The device of claim 1, further comprising one or more fibers intercalated in the frame to provide support.

11. The device of claim 1, further comprising a hub positioned at the apex and in operable communication with the one or more ports.

12. The device of claim 11, wherein the frame comprises a wire, a polymer, a shape memory material, a metal, an alloy, a composite or a combination thereof.

13. The device of claim 1, further comprising a second support structure positioned between the one or more passive chambers and the one or more inflatable active pockets.
